(19) United States
(12) Patent Application Publication
(10) Pub. No.: US 2014/0220572 A1
(43) Pub. Date: Aug. 7, 2014

(54) METHOD AND SYSTEM TO DETECT, DIAGNOSE, AND MONITOR THE PROGRESSION OF ALZHEIMER'S DISEASE

(71) Applicant: Banner Health, Sun City, AZ (US)

(72) Inventors: Diego Mastroeni, Surprise, AZ (US); Joseph Rogers, Glendale, AZ (US); Andrew Grover, Peoria, AZ (US); Paul D. Coleman, New River, AZ (US)

(21) Appl. No.: 14/225,155

(22) Filed: Mar. 25, 2014

Related U.S. Application Data
(63) Continuation of application No. 12/797,591, filed on Jun. 9, 2010, now abandoned.
(60) Provisional application No. 61/185,344, filed on Jun. 9, 2009.

(51) Int. Cl. C12Q 1/68

(52) U.S. Cl. CPC C12Q 1/6883
USPC 435/6.11

(57) ABSTRACT

Various embodiments provide methods for the detection, the diagnosis, and/or the progression monitoring of Alzheimer's disease by observing the epigenetic markers in leukocytes. Methods for determining a state of Alzheimer's disease are provided. Accordingly, these methods can comprise the steps of placing a sample comprising at least one blood component onto a substrate labeling the sample to identify at least one epigenetic marker; determining an amount of the at least one epigenetic marker; comparing the amount to a reference value; and determining a state of Alzheimer's disease.

### CROSS REFERENCE TO RELATED APPLICATIONS

This application is a continuation of U.S. Nonprovisional patent application Ser. No. 12/797,591, filed Jun. 9, 2010, which claims the benefit of U.S. Provisional Patent Application Ser. No. 61/185,344, filed Jun. 9, 2009, and incorporates the disclosure of each application in its entirety by reference. To the extent that the present disclosure conflicts with any referenced application, however, the present disclosure is to be given priority.

### BACKGROUND

Dementia and senility were once accepted as part of the natural aging process. In 1906, Dr. Alois Alzheimer reported histopathologic changes that he had found during the post-mortem examination of a patient suffering from senile dementia. Those changes are recognized today as the neurofibrillary tangles and amyloid plaques that are the hallmarks of Alzheimer's disease. Alzheimer's disease is characterized by progressive neurodegeneration ultimately resulting in dementia and death.

Today, while the ultimate pathology of Alzheimer's disease is fairly well established, effective diagnostic methods and treatment modalities remain elusive because of the complex biological basis for the etiology and pathogenesis of the disease. Scientists and clinicians lack reliable diagnostic tests due to the absence of biologically specific screening techniques. Clinical diagnostic techniques for Alzheimer's disease currently rely on screening individuals displaying symptoms of dementia by excluding other possible causes such as depression, poor nutrition, other dementing conditions (e.g., Parkinson's disease with dementia), or drug interactions. These qualitative and unspecific methods often leave Alzheimer's disease misdiagnosed or unrecognized until later stages in the disease when treatments may be less effective. Early detection and treatment of Alzheimer's disease continues to be the best hope for successful treatment that may delay symptoms and extend a patient's quality of life. Without effective biological and laboratory based diagnostic modalities, the ability to detect and treat Alzheimer's disease in its early stages will remain elusive.

### SUMMARY

Various embodiments provide methods for the detection, the diagnosis, and/or the progression monitoring of Alzheimer's disease by observing the epigenetic markers in leukocytes. Methods for determining a state of Alzheimer's disease are provided. Accordingly, these methods can comprise the steps of placing a sample comprising at least one blood component onto a substrate labeling the sample to identify at least one epigenetic marker; determining an amount of the at least one epigenetic marker; comparing the amount to a reference value; and determining a state of Alzheimer's disease.

Further areas of applicability will become apparent from the description provided herein. It should be understood that the description and specific examples are intended for purposes of illustration only and are not intended to limit the scope of the present teachings.

### BRIEF DESCRIPTION OF THE DRAWINGS FIGURES

The drawing figures described herein are for illustration purposes only and are not intended to limit the scope of the present teachings in any way. The present teachings will become more fully understood from the detailed description and the accompanying drawing figures wherein:

FIG. 1 is a photomicrographic representation of physiologic data relating to the presence of the epigenetic marker 5-methylcytosine in various peripheral blood leukocytes, according to various embodiments of the present invention;

FIG. 2 is a photomicrographic representation of physiologic data relating to changes in the presence of the epigenetic marker 5-methylcytosine in peripheral blood leukocytes of a patient with Alzheimer's disease as compared to a control, according to various embodiments of the present invention;

FIG. 3 is a photomicrographic representation of physiologic data relating to changes in the presence of the epigenetic marker DOC1 in peripheral blood leukocytes of patients with Alzheimer's disease as compared to controls, according to various embodiments of the present invention;

FIG. 4 is a photomicrographic representation of physiologic data relating to changes in the presence of the epigenetic marker MBD2 in peripheral blood leukocytes of patients with Alzheimer's disease as compared to controls, according to various embodiments of the present invention;

FIG. 5 is a photomicrographic representation of physiologic data relating to changes in the presence of the epigenetic marker DNMT1 in peripheral blood leukocytes of patients with Alzheimer's disease as compared to controls, according to various embodiments of the present invention;

FIG. 6 is a bar graph illustrating clinical and physiologic data relating to the quantification of changes in the presence of the epigenetic marker HDAC1 in peripheral blood leukocytes of patients with various clinically diagnosed neurological conditions, according to various embodiments of the present invention;

FIG. 7 is a table illustrating clinical and physiologic data relating to the sensitivity and specificity of correlating changes in the presence of various exemplary epigenetic markers in peripheral blood leukocytes to a clinical diagnosis of Alzheimer's disease, according to various embodiments of the present invention;

FIG. 8 is a photomicrographic representation of clinical and physiologic data relating to changes in the presence of the epigenetic marker 5-methylcytosine in peripheral blood leukocytes of patients exhibiting one of Mild Cognitive Impairment, Alzheimer's disease, or non-demented normal elderly controls, according to various embodiments of the present invention;

FIG. 9 is a bar graph illustrating clinical and physiologic data relating to changes in the presence of the epigenetic marker DNMT1 in peripheral blood leukocytes of patients exhibiting one of Mild Cognitive Impairment, Alzheimer's disease, or non-demented normal elderly controls, according to various embodiments of the present invention;

FIG. 10 is a diagram illustrating a quantitative dot blot, for methylene blue and a calibration curve for methylene blue, according to various embodiments of the present invention; and

FIG. 11 is a diagram illustrating a quantitative dot blot for 5-methylcytosine and a calibration curve for 5-methylcytosine, according to various embodiments of the present invention.

### DETAILED DESCRIPTION

The following description is merely exemplary in nature and is not intended to limit the present teachings, applications, or uses. It should be understood that throughout the drawing figures, corresponding reference numerals indicate like or corresponding parts and features. The description of specific examples indicated in various embodiments of the present teachings are intended for purposes of illustration only and are not intended to limit the scope of the teachings disclosed herein. Moreover, recitation of multiple embodiments having stated features is not intended to exclude other embodiments having additional features or other embodiments incorporating different combinations of the stated features.

Various embodiments provide methods, apparatus, systems and kits for detecting, diagnosing, and/or monitoring the progression of Alzheimer's disease (hereinafter “AD”). A detailed description of various embodiments, namely a method and system for detecting, diagnosing, and monitoring the progression of AD, is provided as to specific enabling disclosure that may be generalized to any application of the disclosed methods and systems in accordance with various embodiments of the present invention. Furthermore, the detailed description of various embodiments includes the best mode known to the inventors at the time of filing this application.

The present invention relates to detecting, diagnosing, and monitoring the progression of AD through epigenetic changes in blood components, such as leukocytes. Leukocytes may comprise any leukocyte subtype such as lymphocytes, neutrophils, basophils, and macrophages. In a representative embodiment of the present invention, a method may comprise detecting epigenetic changes in leukocytes, such as DNA methylation.

In accordance with various embodiments of the present invention, levels of DNA methylation may be decreased in the leukocytes of patients with AD. In an exemplary embodiment, decreases in DNA methylation may be detected, leukocyte of patients in the early stages of AD, where the disease has not yet manifested to the degree that it may be diagnosed using the conventional methods of diagnosis.

The differential diagnosis of neurologic disorders such as AD may comprise performing a variety of conventional methods of diagnosis for elucidating the cause of mental impairment rhea symptoms become apparent. For example, conventional methods of diagnosis may comprise the performance of various qualitative tests by a clinician such an evaluation of a patient's problem solving skills, attention span, counting skills, and memory to determine whether damage has occurred to specific areas of the brain. Further, may systematically rule out causes of the mental impairment by investigating a patient's medical history, such as for indications of previous trauma, family history of neurological disorders, medications, and psychosocial history, such as martial status, living conditions, employment, sexual history; and important life events that may indicate psychological causes such as depression. Through a process of elimination for alternative causes of the mental impairment or dementia, a clinician may begin to suspect AD.

AD cannot be definitely diagnosed until brain tissue is examined after death for the presence of neurofibrillary tangles and amyloid plaques. While the examination of a living patient's brain tissue is not generally feasible or ethical, some microscopic changes to the brain in the later stages of AD may be detected using other conventional methods of diagnosis such as Computed tomography (CT) scanning. Nuclear Magnetic Resonance Imaging (MRI), and Positron Emission Tomography (PET). CT, MRI, and PET techniques may show changes in the brain that are characteristic of late stage AD such as atrophy of the brain, changes in brain activity, and blood vessel structure. Consequently, such techniques cannot detect early stages of the disease where changes remain on a biochemical level inside the neuronal cells of brain tissue.

Expression changes in thousands of genes, spanning multiple biologic pathways, have been reported in pathologically-vulnerable regions of the AD brain. For example, changes to the molecular pathways for energy metabolism, inflammation, and cell cycle regulation have been reported that are believed to contribute to the pathogenesis of AD. These changes in gene expression are widespread in AD, but lack the elucidation of a common over-arching, principle explaining the modification of gene expression across many different seemingly unrelated molecular pathways.

Epigenetic mechanisms may account for or contribute to modulating global gene expression in a cell across different pathways. For example, epigenetic mechanisms causing changes to chromatin or DNA expression such as historic modification, binding of non-histone proteins, or DNA methylation, may be capable of causing global changes to gene expression that may be specific to AD. Epigenetic mechanisms may orchestrate widespread changes in cell phenotype by modifying the transcription of genes involved in many biological pathways across a genome.

Epigenetic mechanisms may involve changes in the micro- and macro-structure of chromatin, as complex of DNA, chromosome proteins, and histone proteins in which the historic proteins are tethered together, in structures round which double-stranded DNA is wound. Conformational changes in historic proteins or modifications of the way in which DNA wraps around the histories may then differentially alter access of the transcriptional machinery to some genes while leaving access to other genes intact.

Although there are multiple mechanisms by which histones are modified, including methylation, phosphorylation, ubiquitination, sumoylation, citruilination, ADP-ribosylation, and other post-translational modifications of the amino acids that make up histone proteins, histone acetylation is one of the most ubiquitous and well studied. Histone acetyltransferases (HATs) catalyze the transfer of an acetyl group from acetyl-coenzyme A to lysine residues on the N-termini of histone proteins. As a result of acetylation, the positive charge of the historic proteins is neutralized, decreasing interactions of the histone protein tails negatively-charged phosphate groups of associated DNA. This conformational relaxation of the chromatin permits access to and transcription of genes within the complex. Conversely, the histone deacetylases (HDACs) transfer acetyl groups from acetylated historic proteins back to coenzyme producing more condensed chromatin state and decreased or silenced gene transcription.

DNA methylation comprises one type of epigenetic mechanism that modifies DNA, resulting in changes in gene expression. Adjacent cytosine-guanine dinucleotides (CpGs) within DNA sequences may be methylated by proteins called DNA methyltransferases. Methylation of cytosine-guanine dinucleotide pairs (CpGs) may inhibit the access of the cell's transcriptional machinery to the promoter region of the gene containing the methylated CpG sequence. Methylation may occur within the coding region of a gene or in repetitive DNA sequences that may flank a gene. Such methylation may alter gene expression even if it occurs at some distance from the promoter region.

Highly methylated genes may exhibit a decrease or repression in gene expression. Conversely, mechanisms that demethylate CpGs leading to hypomethylated DNA, may lead to an upregulation of gene expression. However, these trends are not universal as exceptions have been found in which genes that are hypomethylated exhibit repressed gene expression and genes that are hypermethylated are upregulated. Consequently, the expression of a particular gene and resulting changes in protein levels must be assayed to verify the effect of methylation on that particular gene.

DNA methylation is highly interactive with histone acetylation and the other histone-modifying mechanisms. Adjacent CpGs within DNA can be methylated by the actions of the DNA methyltransferases, DNMT1, DNMT2, DNMT3a/b, and DNMT4. In mammals, DNMT1 appears to be primarily involved in maintenance methylation of hemimethylated DNA after DNA replication, whereas DNMT3a and DNMT3b are involved in de novo methylation. DNMT2 is typically considered to be an RNA methyltransferase, although it also has 5-cytosine DNA methyltransferase activity and forms denaturant-resistant complexes with DNA. The methyl group that is transferred to cytosine by the DNMTs ultimately derives from folate through its interactions with S-adenosylmethionine and, further upstream, the homocysteine-methionine cycle.

Through these processes, approximately 70% of CpG dinucleotides within the human genome are methylated. Although methylation can take place at any CpG site on a gene, it may be particularly important with respect to CpG-rich stretches (CpG islands) within the promoter region. Some 50,267 CpG islands exist in the human genome, with 28,890 in simple repeat and low complexity sequences that are masked.

A second, linked mechanism by which DNA methylation may modify gene expression is through methyl-cytosine-binding complexes (MeCPs) such as MeCP2. When bound to methylated DNA, MeCP2 has been shown to recruit HDACs, which, as noted earlier, ray then induce a more condensed chromatin state and decreased or silenced gene transcription. Mutations of the MeCP2 gene cause Rett's Syndrome, with dysregulation of neural development, mental retardation, and motor dysfunction.

MeCP1, a macromolecule made up of some 10 different peptides, including DOC1, may also act as a mediator between methylation and histone acetylation, recognizing and binding to CpG dinucleotides, recruiting HDACs, and inducing transcriptional repression. Unlike MeCP2, however, MeCP1 dots not bind directly to methylated DNA, but to a single methyl-CpG-binding domain protein, MBD2, in addition to inducing histone modifications, MBD2-bound MeCP1 helps maintain the methylation status of CpGs by recruiting DNMT1. DNMT1 is then able to recognize and repair CpGs that have lost methyl groups on one DNA strand but not the other.

The epigenetic mechanisms discussed above may be considered in terms of epigenetic markers. As known to those skilled in the art, the term “marker” is generally accepted as any specific character that may be detected by a biochemical test, or an analytical test, or a combination thereof. For example, a marker may indicate a presence or absence of an enzyme in a sample, and in some cases the marker may be used to determine a concentration of the enzyme in the sample. Also, a marker may indicate, for example, an activity of a biochemical reaction in a sample. Still further, a marker may indicate for example, a presence or absence of protein in a sample, and in some cases the marker may be used to determine a concentration of the protein in the sample. As used herein, the term “epigenetic marker” is defined as at least one of a DNA methylation marker and a histone modification marker. Examples of a DNA methylation marker include, but are not limited to, 5-methylcytosine, 5-methylcytidine, DNMT1, DNMT2, MeCP, DOC1, MBD2, and MBD3. Examples of a histone modification marker include, but are not limited to, HDAC1, HDAC2, and HAT.

DNA methylation was once studied in the context of maintaining DNA methylation during cell divisions. However, the role of DNA methylation has been elucidated in postmitotic cells, including neurons in the field of neuroepigenetics. Neuroepigenetic studies of DNA methylation illustrate its role in mediating neuronal and synaptic plasticity, such as long-lasting modifications to hypothalamic neurons causing physiologic, memory, and behavioral changes in mice resulting from stress in early life.

The brain tissue of patient's with Alzheimer's disease known to be vulnerable to damage by the disease, such as entorhinal cortex layer II neurons, exhibits marked decreases in immunoreactivity for markers of DNA methylation and DNA methylation maintenance factors. For example, labeling neurons with an antibody to 5-methylcytosine and 5-methylcytidine, which are markers for methylated DNA, reveals dramatic decreases in immunoreactivity in brain tissue samples from patient's with AD compared to samples from patient's without the disease.

The development of a diagnostic method for Alzheimer's disease based on the decreased incidence of markers in brain tissue is impractical for a variety of reasons such as the invasiveness and procedural risk of obtaining brain tissue and its associated high cost. Detecting the disease in its earliest stages makes such an approach highly problematic as the patient may be exhibiting only vague symptoms of the disease or no symptoms at all, which may make the procedural risk and cost of diagnosis unjustifiable.

The development of a noninvasive diagnostic technique for evaluating epigenetic changes characteristic of AD is thus problematic and infeasible in brain tissue. Basing the diagnostic technique on easily obtainable biological samples that may be collected at routine doctor's visits, such as blood, urine, a mouth swab, or a hair sample presents advantages in terms of convenience, cost control, and reduced procedural risk. However, according to the literature and other medical sources, no such diagnostic techniques currently exist.

As disclosed herein unexpected and surprising results have been obtained. The inventors have developed methodology to determine the disease state of AD by analyzing a blood sample from a patient. These surprising and unexpected results are related to the discovery that global DNA methylation level in leukocytes of a blood sample can be related to a disease state of AD in a patient with extremely high specificity as compared to other diseases or a non AD state.

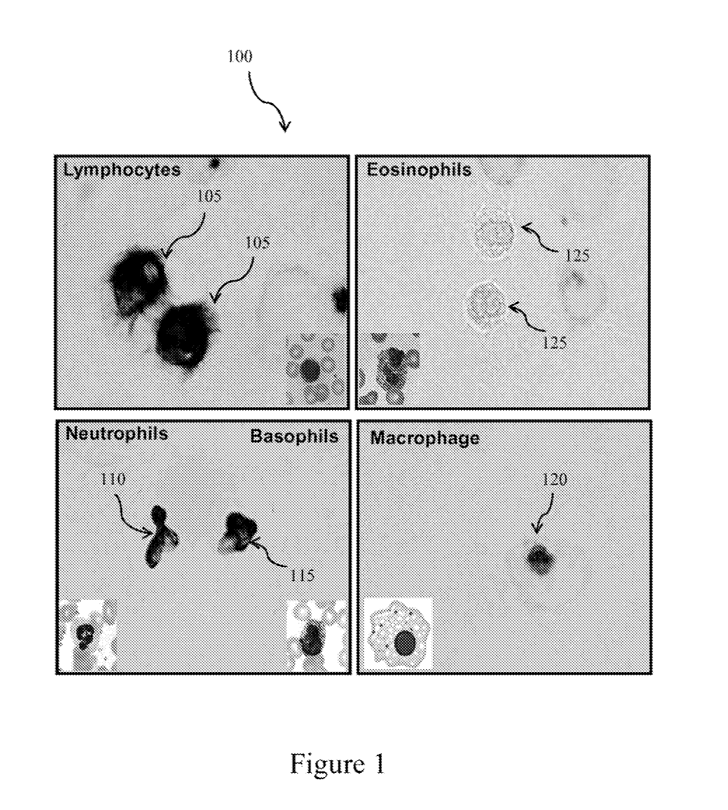

Now with reference to FIG. 1, a photomicrographs 100 illustrating physiologic data relating to the presence of the epigenetic marker methylcytosine in various peripheral blood leukocytes, according to various embodiments of the present invention. According to various embodiments of the present invention, an immunoassay can be performed on a sample of peripheral blood leukocytes from a cognitively normal elderly patient. The immunoassay can comprise the application of the isolated leukocytes to a substrate, such as for example, microscope slide followed by treatment with primary antibodies to 5-methylcytosine. Excess primary antibody can be washed away, which is followed by the application of a reporter molecule conjugated secondary antibody. A colored signal can be developed and observed in the leukocyte cells on the substrate using a microscope. Lymphocytes 105, neutrophils 110, basophils 115, and macrophages 120 can exhibit immunoreactivity to the antibodies, indicating the presence of 5-methylcytosine, as illustrated in FIG. 1. However, eosinophils 125 may not show immunoreactivity to antibodies binding 5-methylcytosine under these conditions.

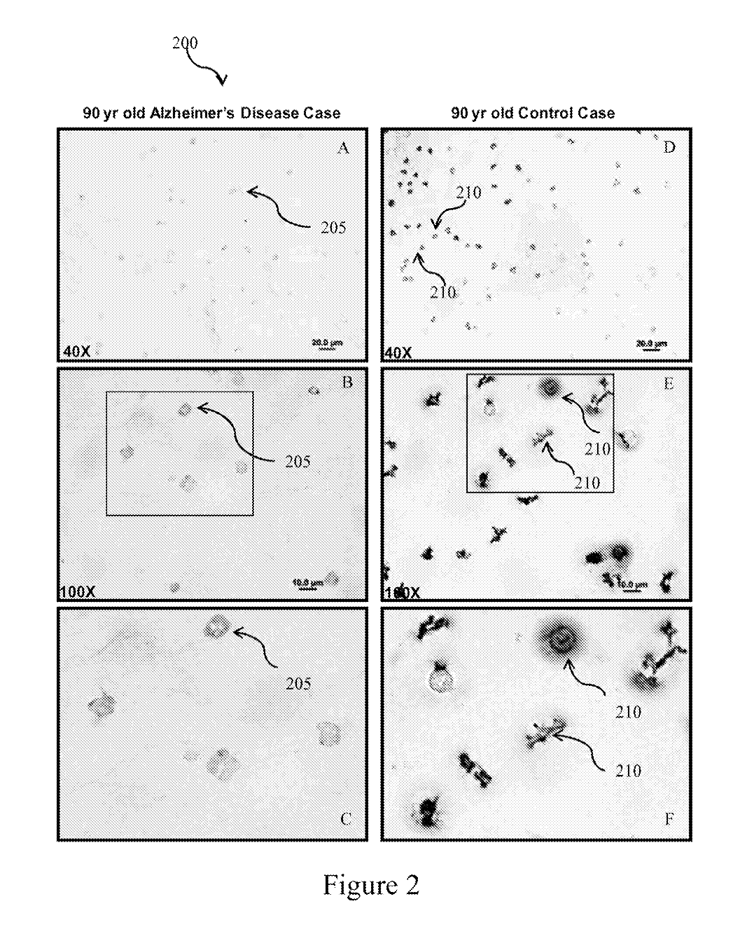

Moving to FIG. 2, photomicrographs 200 illustrate physiologic data relating to changes in the presence of the epigenetic marker 5-methylcytosine in peripheral blood leukocytes of a patient with Alzheimer's disease as compared to a control, according to various embodiments of the present invention. In accordance to various embodiments, an immunoassay using primary antibodies to 5-methylcytosine can be performed on samples of peripheral blood leukocytes from a cognitively normal 90 year old patient (as illustrated in right column) and a 90 year old patient diagnosed with AD (as illustrated in left column). As illustrated in FIG. 2, Micrographs 200 show that leukocytes 205 from the 90 year old patient diagnosed with AD exhibit decreased immunoreactivity compared to the leukocytes 210 of a cognitively normal 90 year old patient. Panels (a) and (b) are exemplary micrographs illustrating stained leukocytes shown at 40× magnification, with panels (c) and (d) showing the same exemplary micrographs at 100× magnification. Panels (e) and (f) illustrate further enlargements of the boxed areas indicated in panels (c) and (d), respectively.

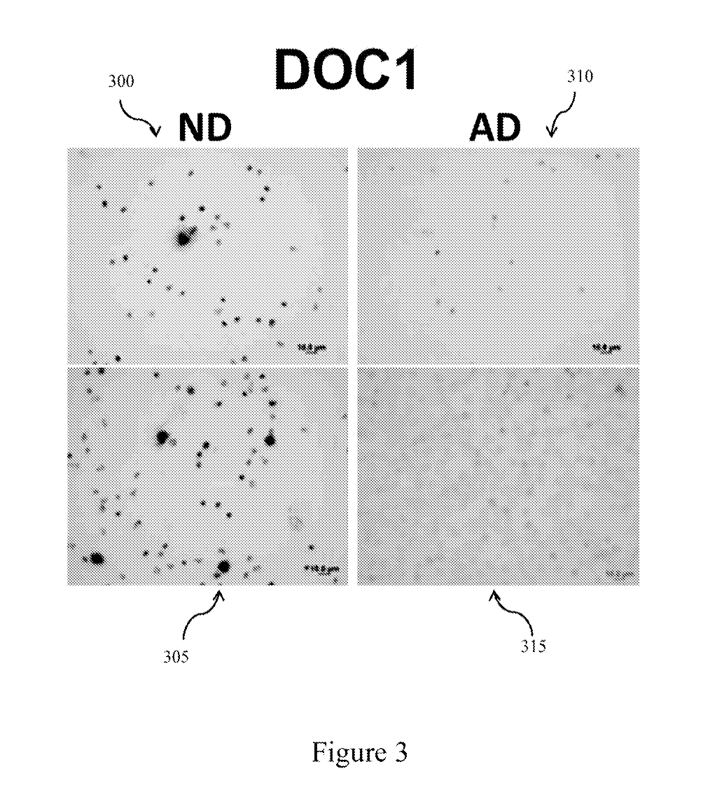

Referring to FIG. 3, photomicrographs 300 illustrate physiologic data relating to changes in the presence of the epigenetic marker DOC1 in peripheral blood leukocytes of patients with Alzheimer's disease as compared to controls, according to various embodiments of the present invention. As illustrated and in accordance with various embodiments, differences between the immunoreactivity of leukocytes isolated from two patients diagnosed with AD by conventional diagnostic methods and two non-diseased (ND) elderly control patients to antibodies for the epigenetic marker DOC1 can be observed. The immunoassay using primary antibodies to DOC1 can be performed on leukocytes from each of the patients. As illustrated, the leukocytes 310, 315 from patients with AD exhibited decreased immunoreactivity to the antibody for DOC1 compared to the leukocytes 300, 305 from the ND patients, thus signifying a decreased amount of DOC1 present in the leukocyte cells for patients with AD.

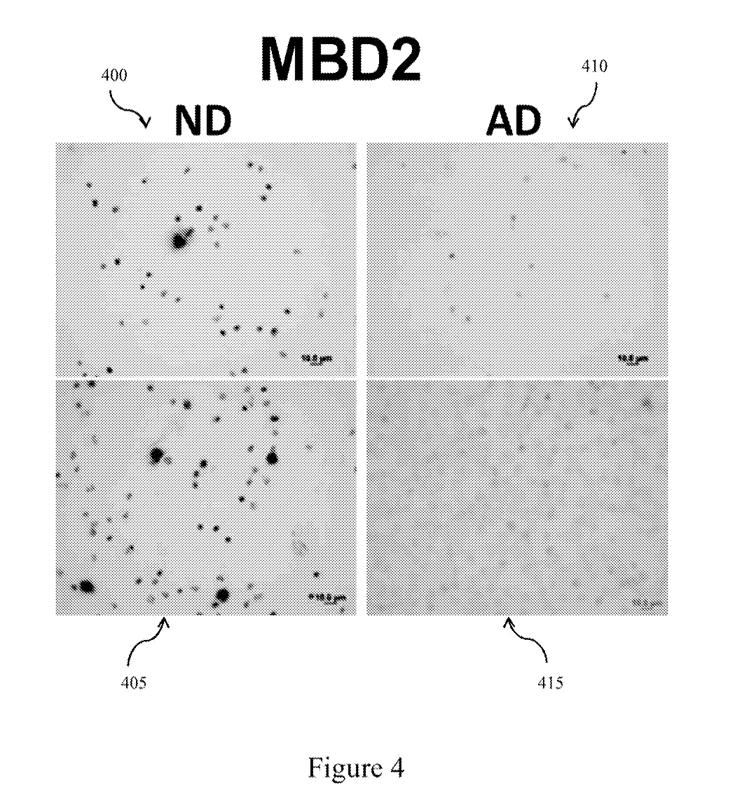

Now turning to FIG. 4, photomicrographs 400 illustrates physiologic data relating to changes in the presence of the epigenetic marker MBD2 in peripheral blood leukocytes of patients with Alzheimer's disease as compared to controls, according to various embodiments of the present invention. The immunoassay using primary antibodies to the epigenetic marker MBD2 can be performed on leukocytes from each of two patients diagnosed with AD by conventional diagnostic methods and two ND control patients. As illustrated, the leukocytes 410, 415 from patients with AD exhibited decreased immunoreactivity to the antibody for MBD2 compared to the leukocytes 400, 405 from the ND patients, thus signifying, a decreased amount of MBD2 present in the leukocyte cells for patients with AD.

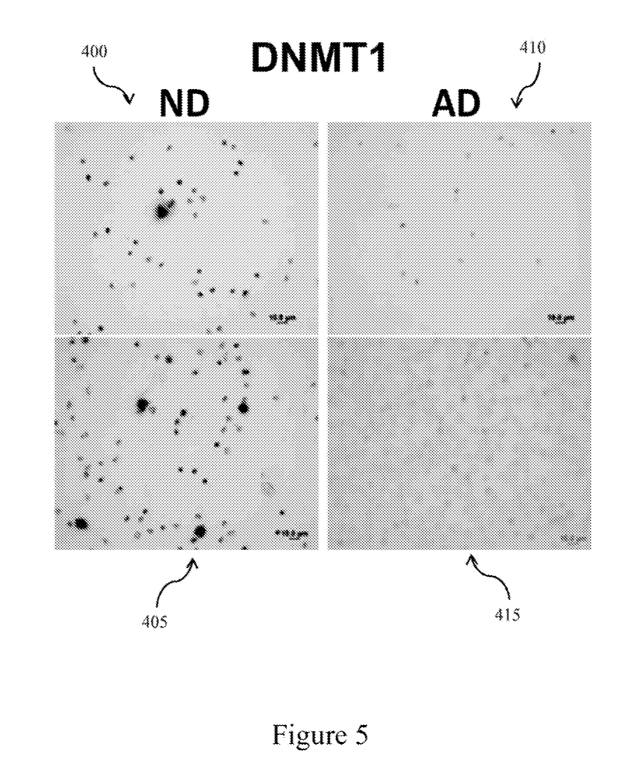

Referring to FIG. 5, photomicrograph 500 illustrate physiologic data relating to changes in the presence of the epigenetic marker DNMT1 in peripheral blood leukocytes of patients with Alzheimer's disease as compared to controls, according to various embodiments of the present invention. The immunoassay using primary antibodies to the epigenetic marker DNMT1 can be performed on leukocytes from each of two patients diagnosed with AD by conventional diagnostic methods and two ND control patients. As illustrated, the leukocytes 510, 515 from patients with AD exhibited decreased immunoreactivity to the antibody from DNMT1 compared to the leukocytes 500, 505 from the ND patients, thus signifying a decreased amount of DNMT1 present in the leukocyte cells for patients with AD.

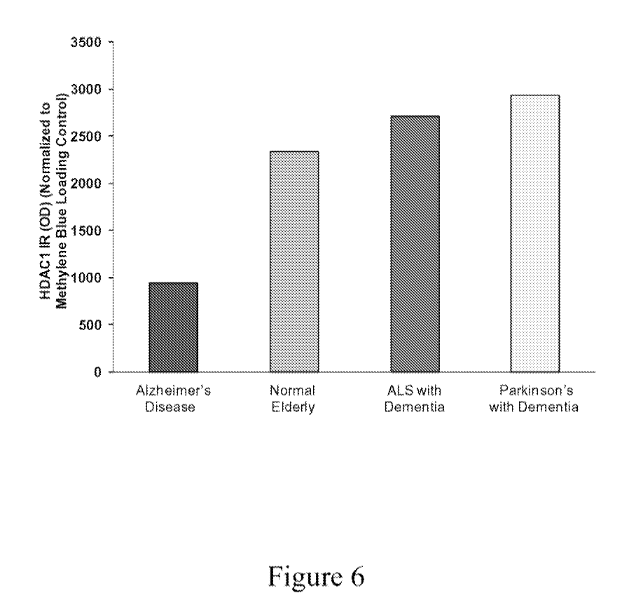

Referring to FIG. 6, a bar graph illustrating clinical and physiologic data relating to the quantification of changes in the presence of the epigenetic marker HDAC1 in peripheral blood leukocytes of patients with various clinically diagnosed neurological conditions, according to various embodiments of the present invention. In accordance to various embodiments, the immunoreactivity of peripheral blood leukocytes to antibodies that bind an epigenetic marker from patients with various neurological conditions can be quantified. Levels of the epigenetic marker can be quantified by performing a dot blot assay for protein in which a nitrocellulose membrane is spotted with cell lysate from leukocytes containing the cell's protein. The cell lysate was dried onto the membrane with vacuum dot blot manifold. The membrane can be incubated with a primary antibody to the epigenetic marker, washed, and then treated with a reporter molecule conjugated secondary antibody. A colored signal can be developed and its intensity was measured using a densitometer configured to measure the optical density of colored substrate on the membrane.

As illustrated in FIG. 6, the immunoreactivity of peripheral blood leukocytes to antibodies that bind the epigenetic marker HDAC1 from patients with various neurological conditions can be quantified. Levels of HDAC1 can be quantified by performing a dot blot assay for protein, as discussed herein. Measurements of the levels or the optical density can be normalized to the optical density of a β-actin loading control. The normalized optical density of the signal from the secondary antibody in the sample of patients diagnosed with AD is approximately 30% of the signal from the sample of patients diagnosed with Parkinson's disease. The intensity of the sample of patients diagnosed with AD is approximately 36% of the signal from the sample of patients diagnosed with Amyotrophic Lateral Sclerosis with dementia and approximately 40% of the signal from sample of patients being ND controls. As illustrated, a marked decrease in signal for; immunoreactivity to HDAC1 antibodies in AD leukocyte samples compared to leukocyte samples derived from patients with other neurological conditions or patients that lack disease is observed which can distinguish AD from other conditions.

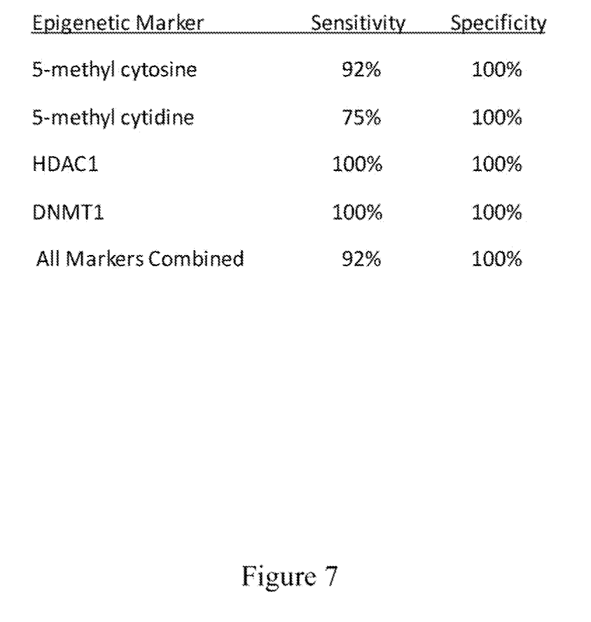

FIG. 7 is a table illustrating clinical and physiologic data relating to the sensitivity and specificity of correlating changes in the presence of various exemplary epigenetic markers in peripheral blood leukocytes to a clinical diagnosis of Alzheimer's disease, according to ‘various’ embodiments. Of the present invention. In accordance with various embodiments, peripheral blood samples were obtained from 51 patients that were diagnosed with Alzheimer's disease by conventional diagnostic methods, patients with other neurological conditions such as Parkinson's disease, and normal elderly control patients. Samples were assayed for immunoreactivity to antibodies using, the immunoassay with primary antibodies to the epigenetic markers 5-methylcytosine, 5-methylcytdine, HDAC1, and DNMT1. 100% specificity for detecting AD was observed such that the decrease in the level of the epigenetic marker resulted in a determination of AD for every patient that was diagnosed with AD by conventional diagnostic methods. Diagnosis based on the epigenetic markers also showed 75% to 100% sensitivity for detecting AD such that the levels of the epigenetic markers present in each leukocyte sample discriminated between patients with AD and patients with other neurological conditions. As illustrated herein, various embodiments of the present invention can include analyzing as plurality of epigenetic markers in leukocytes from patient sample and further can include determining a disease state from the resulting analysis of the plurality of epigenetic markers.

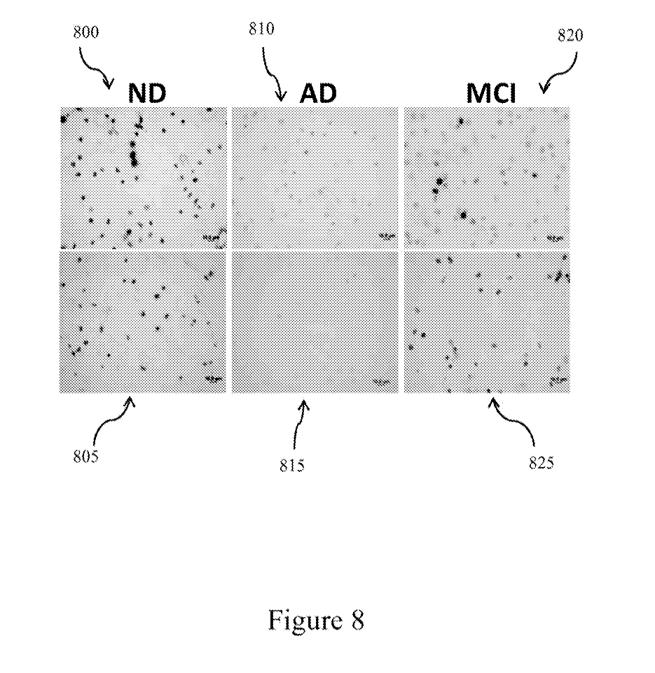

Referring to FIG. 8, photomicrographs illustrate clinical and physiologic data relating to changes in the presence of the epigenetic marker 5-methylcytosine in peripheral blood leukocytes of patients exhibiting one of Mild Cognitive Impairment, Alzheimer's disease, or non-demented normal elderly controls, according to various embodiments of the present invention. Immunoreactivity to antibodies binding the epigenetic marker 5-methylcytosine using the immunoassay can be observed in patients diagnosed with one of Mild Cognitive Impairment (MCI) or AD. MCI may be clinically diagnosed where patient's daily activities are not affected, but the patient may experience impairment with memory, language, attention, reasoning, judgment, reading, and writing. Patient's with MCI are considered to be at high risk for progressing from normal cognition to the dementia of Alzheimer's disease, with 30-40% of MCI patients being formally diagnosed with Alzheimer's disease within three years, particularly where the primary impairment is with memory.

As illustrated in FIG. 8, Control leukocytes 800, 805 from two patients with normal cognition exhibit positive immunoreactivity to antibodies binding 5-methylcytosine, representing normal. DNA methylation. Leukocytes 810, 815 from two patients with AD exhibit a marked decrease of immunoreactivity of 5-methylcytosine. However, leukocytes 820, 825 from two patients diagnosed with MCI exhibit an intermediate immunoreactivity to 5-methylcytosine antibodies. The intermediate level of immunoreactivity is indicative of is repression or abnormal amount of DNA methylation that may eventually approach the decreased levels of DNA methylation observed in AD.

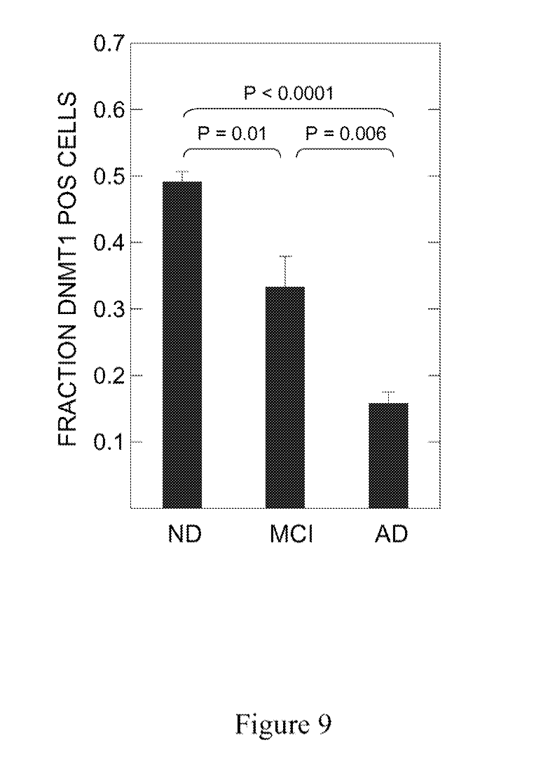

Referring, to FIG. 9, a bar graph illustrates clinical and physiologic data relating to changes in the presence of the epigenetic marker DNMT1 in peripheral blood leukocytes of patients exhibiting one of Mild Cognitive Impairment, Alzheimer's disease, or non-demented normal elderly controls, according to various embodiments of the present invention. Immunoreactivity to antibodies binding the epigenetic marker DNMT1 in patients diagnosed with MCI can be observed. Peripheral blood leukocytes were isolated from patients diagnosed with MCI or AD by conventional diagnostic methods and from ND controls. The immunoassay can be performed on the leukocyte samples with primary antibody to DNMT1. The slides were observed blind using a microscope where the total number of cells with visual immunoreactivity to the DNMT1 antibodies were counted. This number was divided by the total number of cells on the slide to provide a fraction of positively immunoreactive cells. Approximately 50% of the cells from ND control samples were immunoreactive, indicating a normal amount of DNMT1 for DNA methylation. However, only approximately 16-18% of AD samples were immunoreactive, indicating a decreased amount of DNMT1 available for DNA methylation. An intermediate level of DNMT1 antibody immunoreactivity was observed for MCI samples, indicating a decrease in the amount of available DNMT1, but not as low as the AD levels.

In accordance with exemplary embodiments and with reference to the Figures discussed above, two exemplary concepts are shown. First, differences in DNA methylation in AD, ND, and MCI patients are so dramatic that they can be seen with the naked eye. Second, the multiple markers related to DNA methylation all show this difference. DNA methylation is a complex process. These finding indicate that not only is DNA methylation itself profoundly altered in AD, but also that there are equally profound deficits in the molecules that perform and maintain DNA methylation.

Hence, in various embodiments, any epigenetic marker or a combination thereof can be used to determine a disease state of AD, including a 5-methyl cytosine marker, a DNMT1 marker, a HDAC1 marker and 5-methyl cytidine marker. In various embodiments, 5-methyl cytosine and 5-methyl cytidine can provide direct measures of DNA methylation. Although, DNMT1 and HDAC1 are not direct measures of DNA methylation, these exemplary markers can still be used to determine a disease state of AD. DNMT1 is a molecule that performs the methylation and HDAC1 is a molecule that mediates changes in histones as a result of changes in DNA methylation. In various embodiments, epigenetic markers can include hut not limited to markers such as DOC1, DNMT2, DNMT3a/b, HDAC2, MBD2, MBD3, RPL26, p66, MTA2, RbAp48, and combinations thereof.

Various embodiments of the present invention provide methods for the detection, the diagnosis, and/or the monitoring of the progression of AD by observing a present state of the global DNA methylation of leukocytes in a patient sample. According to various embodiments, present state of the global DNA methylation of leukocytes can be determined by either a direct measure of global DNA methylation or a measure of at least one epigenetic marker linked to global DNA methylation, including histone-related markers. An exemplary method can comprise the steps of collecting a blood sample; isolating leukocytes or a portion thereof from the blood sample; binding an antibody to at least one epigenetic marker located in the leukocytes; staining or otherwise labeling the antibody bound to the epigenetic marker, observing, or measuring, or quantifying an amount of stain or a signal from a label bound to the at least one epigenetic marker; and comparing the amount of stain or the signal from the label to a qualitative or quantitative reference value.

In exemplary embodiments of the methods, the stain or the label can comprise any moiety that can conjugate to an antibody that hinds to an epigenetic marker, such as for example, a methylated DNA site or to an epigenetic mechanism of DNA methylation, such as, for example but not limited to, methylation promoters, methylation inhibitors, methylation maintainers, and histone-related markers. Still further, in other exemplary embodiments of the methods, the stain or the label can comprise an antibody that binds to an antibody that binds to an epigenetic marker. Moreover, in various embodiments, the epigenetic marker is at least one of a DNA methylation marker and a histone modification marker. Examples of the at least one epigenetic marker can include but are not limited to 5-methylcytosine, 5-methylcytodine, DOC1, DNMT1, DNMT2, DNMT3a/b, HDAC1, HDAC2, HAT1, MBD2, MBD3, RPL26, p66, MTA2, RbAp48, and combinations thereof.

The methods can include the addition of a label, such as a visible dye or fluorophore conjugated to a detecting secondary antibody for subsequent visualization or observation. For example, such a label may be visualized or observed by a human eye, with magnification, such as for example an optical microscope or without magnification. In another example, such a label may be visualized or observed by use of to reader such as, for example but not limited to, a spectrometer, a flourometer, a fluorescence detector, a colorimeter, a densitometer, flow cytometer, an immunosorbent assay or other techniques that are familiar to those skilled in the art or are created in the future. However, any method of visualization or observation can be largely dependent on the stain or the label that is chosen.

According to various embodiments of the present invention, an immunoassay can be used to analyze a sample comprising a leukocyte or protein or DNA extract from a leukocyte and determination of an amount of at least one epigenetic marker. The particular format of the immunoassay of the present invention is not critical to the present invention. Examples of such formats include an ELISA, radio-immunoassay, dot blot assay, slot blot assay, immunoprecipitation and protein quantification, immuno-PCR, and Western blot.

As described herein. DNA methylation is an epigenetic event that refers to the covalent addition of a methyl group, catalyzed by a family of DNMT enzymes, to the 5-carbon of cytosine in a CpG dinucleotide. Methods for DNA methylation analysis can be divided roughly into two types: global and gene-specific DNA methylation analysis. According to various embodiments, for global DNA methylation analysis, methods which measure the overall level of methyl cytosines in the genome can include chromatographic methods and methyl accepting capacity assay. For gene-specific DNA methylation analysis, a large number of techniques have been developed. Most early studies used methylation sensitive restriction enzymes to digest DNA followed by Southern detection or PCR amplification Recently, bisulfite reaction based methods have become very popular such as DNA methylation specific PCR (MSP), bisulfite genomic sequencing PCR. Additionally, in order to identify unknown DNA methylation hot-spots or methylated CpG islands in the genome, several genome-wide screening methods have been invented such as Restriction Landmark Genomic Scanning for Methylation (RLGS-M), and CpG island microarray.

Furthermore, a sample comprising a leukocyte can be analyzed by a variety of methods to determine an amount of at least one epigenetic marker including but not limited to fluorescence detection, DNA sequencing gel capillary electrophoresis on an automated DNA sequencing machine, microchannel electrophoresis, and other methods of sequencing, mass spectrometry, time of flight mass spectrometry, quadrupole mass spectrometry, magnetic sector mass spectrometry, electric sector mass spectrometry infrared spectrometry, ultraviolet spectrometry, palentiostatic amperometry or by DNA hybridization techniques including Southern Blots, Slot Blots, Dot Blots, and DNA microarrays, wherein DNA fragments would be useful as both “probes” and “targets,” ELISA, fluorimetry, Fluorescence Resonance Energy Transfer (FRET), SNP-IT, GeneChips, HuSNP, BeadArray, TaqMan assay, invader assay, MassExtend or MassCleave™ (hMC) method.

White blood cell (WBC) or leukocyte isolation from peripheral blood can be accomplished using a wide variety of methodologies, such as for example, but not limited to standard density gradient separation, commercially available evacuated separation tube systems, cell sorting systems, or other techniques familiar to those skilled in the art.

As can be appreciated by those skilled in the art, blood can be fractionated, and the different fractions of the blood can be used for different medical needs. Under the influence of gravity or centrifugal force, blood spontaneously sediments into three layers. At equilibrium, the top low-density layer is a straw-colored clear fluid called plasma. The bottom, high-density layer is a deep red viscous fluid comprising a nuclear red blood cells (erythrocytes) specialized for oxygen transport. The intermediate layer is the smallest, appearing as a thin white band above the erythrocyte layer and below the plasma layer; this is called the huffy coat. The huffy coat itself has two major components, nucleated leukocytes (white blood cells) and a nuclear smaller bodies called platelets (or thrombocytes).

Also, as can be appreciated by those skilled in the art, one as of obtaining white cells from whole blood is simply to allow EDTA-blood to settle in siliconized glasses and then pipette off the leukocyte-rich supernatant. Separating blood to isolate the WBC component or the leukocytes is well known to those skilled in the art. However in various embodiments, whole blood or a portion of blood that comprises leukocytes can be analyzed by methods described herein and without separating the WBC component or the leukocytes from the whole blood or the portion of blood that comprises leukocytes.

In various embodiments, the present invention provides methods for determining a state of AD in a human. Accordingly, exemplary methods can comprise the steps of: placing a sample comprising at least one blood component onto a substrate; labeling the sample to identify at least one epigenetic marker, determining an amount of the at least one epigenetic marker, comparing die amount to a reference value; and determining a state of AD. These exemplary methods can further comprise the step of separating blood into the at least blood component and other blood components, to produce the sample comprising at least one blood component onto a substrate In various embodiments of the exemplary methods, the at least one blood component comprises leukocytes. The sample can be from a patient.

Furthermore, these exemplary methods can comprise the step of preparing a treatment plan for a patient. In addition, these methods can comprise the step of treating the patient with a therapeutic substance. These methods can further comprise the steps of placing a second sample comprising the at least one blood component onto the substrate; labeling the second sample to identify the at least one epigenetic marker; determining a second amount of the at least one epigenetic marker; comparing the second level to the reference value; and further determining a state of AD. An analysis of the second sample can be substantially simultaneous with the sample or the analysis can be later in time after the analysis of the sample. These methods can include the step of determining a dosage of a therapeutic substance to administer to the patient. In various embodiments, the reference value comprises a calibration curve for varying amounts of a label attached to the at least one epigenetic marker. These exemplary methods can comprise a step of observing a quantitative amount of the label. These methods can comprise binding an antibody to at least one epigenetic marker. Still further, these methods can comprise the step of introducing an antibody comprising a label to conjugate to the antibody.

In accordance to various embodiments, any of the methods discussed herein can be stretched over time such as for example, a longitudinal study comparing a first set patient's results related one of more epigenetic markers at a first point in time to a second set of patient's results related one of more epigenetic markers at a second point in time. Such a comparison can provide one of a prediction or likelihood of developing AD. Such a comparison can provide a likely rate of developing AD. Still further, such a comparison can be useful in evaluating an efficacy of a therapeutic substance, as well as adjusting a dosage of such a therapeutic substance. Such a comparison can be part of a treatment plan. Although such results can be calculated by extrapolating from a single point measurement, at least two or more measurements taken some time apart as longitudinal data, would confirm the single point extrapolation provide a new state of Alzheimer's disease. For example, the measurement can be taken from one week to 2 years apart. However, the frequency of measurement could be about every 3 months, or about every 6 months, or about once a year, or about bi-annually, in various embodiments, a comparison can produce a difference (T0−T1) or a rate of change (T0−T1)/Time, where T0=marker amount or value at time zero. T1=amount at lime one, and Time=the amount of time between measurements.

Listed in Table 1 below are commercially available antibodies that may be useful in accordance to various embodiments of the present invention. These commercially available antibodies may be useful in binding to an epigenetic marker in a leukocyte. These commercially available antibodies are specific to an individual epigenetic marker. However, a plurality of these commercially available antibodies or other similar antibodies not listed may be included in kits in accordance with various embodiments.

TABLE 1
Commercial Antibodies

| Antibody | Host/Type | Source/catalogue# | Antigen/Epitope |
|---|---|---|---|
| MBD2 | Goat | Abcam/ab58241 | SEQ ID NO: 1 |
| | polyclonal | | |
| MBD3 | Mouse | Abcam/ab45027 | SEQ ID NO: 2 |
| | monoclonal | | |
| DOC1 | Rabbit | Abcam/ab31794 | SEQ ID NO: 3 |
| | polyclonal | | |
| DNMT1 | Rabbit | Abcam/ab19905 | Within residues |
| | polyclonal | | 100-200 |
| 5- | Mouse | Genway/ | Methylated- |
| methylcytidine | monoclonal | 20-783-71683 | cytosine |
| | | | DNA/RNA |
| HDAC1 | Rabbit | Abcam/ab19845 | residues 450 |
| | polyclonal | | to the |
| | | | C-terminus |
| HDAC2 | Rabbit | Abcam/ab32117 | Residues within |
| | polyclonal | | C-terminal end |
| 5- | Mouse | Aviva Systems | Methylated- |
| methylcytosine | monoclonal | Biology AMM99021 | cytosine |
| | | | DNA |
| Pan methyl | Rabbit | Cell signaling/ | Methylated H3 |
| Histone H3 | monoclonal | 4473 | |

In various embodiments, the present invention provides methods for determining a state of AD in a human patient. Accordingly, exemplary methods can comprise the steps of receiving a blood sample from a patient; separating leukocytes from the blood sample; binding a first antibody to at least one epigenetic marker in the leukocytes; conjugating a second, antibody comprising a label to the first antibody, determining an amount, of the label; and determining the state of AD in the patient based on the amount of the label.

These methods can further comprise the step of adding EDTA to the blood sample, in which the separating the leukocytes can be by gravity. However in non-coagulated blood, the separating the leukocytes can be by centrifuge. As can be appreciated by those skilled in the art, EDTA when added to a blood sample can be at least one of preservative and an anticoagulant.

These exemplary methods can comprise the steps of binding third antibody to a second epigenetic marker in a second portion of the leukocytes, conjugating a fourth antibody comprising a second label to the thud antibody; determining an amount of the second label; and determining the state of AD in the patient based on the amount of the label and the amount of the second label. These exemplary embodiments can comprise the step of comparing the amount of the label to a reference. In various embodiments, the reference can comprise a calibration curve for an epigenetic marker. Moreover, in various embodiments, the at least one epigenetic marker is at least one of a DNA methylation marker and a histone modification marker.

In various embodiments, proteomic techniques using mass spectrometry may be used to identify and quantify a particular protein or peptide, such as an epigenetic marker, in a protein extract derived, from a biological sample. In some embodiments, the epigenetic marker may be identified by comparing the theoretical mass to the mass of the proteins or peptides acquired experimentally in the sample using mass spectrometer. To determine the mass of a protein, its amino acid sequence may be submitted to proteomic software programs that determine the mass of proteins, peptides, and amity) acids. These masses can then be compared to data generated by mass spectrometry analysis. In another embodiment, the sequence of an unknown isolated protein may be obtained by sequencing the protein with conventional amino acid sequencing techniques such as Edman degradation. The proteomic software program may then perform a virtual enzymatic digestion of the protein, such as with the enzyme trypsin, which cleaves proteins at known amino acid sequences, to produce peptide fragments. The resulting peptide fragments when run on a liquid chromatography mass spectrometry (LC-MS) system may produce a specific peptide mass fingerprint (PMF) that specifically identifies the protein it is derived from. In one embodiment, the PMF of an unknown isolated protein may be determined without sequencing by application of the digested protein to the mass spectrometer to determine the mass of its constituent peptides followed by a comparison of the peptide masses to protein database entries.

Once the PMF is obtained for the epigenetic marker, the quantification of the epigenetic marker from an actual biological sample rutty be determined. For example, a protein fraction from cell lysate samples may be digested with proteolytic enzymes that cleave proteins at specific locations. The resulting digested fragments may be introduced into as mass spectrometer by techniques such as matrix-assisted laser desorption and ionization (MALDI) or electrospray ionization (EST-MS). These ionization techniques produce charged species which masses can be filleted and analyzed by mass analyzers, such as time of nights (TOFs), quadrupole, or ion trap, may determine the mass of the peptides. The data acquired by the mass spectrometer in combination with proteomic data analysis software programs can quantify epigenetic marker levels in the samples when used with techniques such as added internal standards or spectral counting.

In various embodiments, antibodies may be used as a probe to identify particular molecules in cells, tissues, and biological fluids such as blood using immunofluorescence microscopy. A primary antibody that binds to a specific antigen, such as an epigenetic marker, may be labeled directly by covalently binding a dye, such as a fluorescent molecule, to the primary antibody. More commonly, the binding of the primary antibody to the antigen may be detected by a secondary antibody labeled with a fluorescent molecule whose antigen is any other antibody. The labeled secondary antibody may be called a fluorescent anti-immunoglobulin. The fluorescent molecule may be excited by light at a particular wavelength, such as blue or green, resulting in the emission of light of a different wavelength for detection. The fluorescent molecule may comprise any number of conventional fluorescent molecules, such as green fluorescent protein from the jellyfish Aequorea Victoria. In an alternative embodiment to fluorescence, immunohistochemistry may be used in which the primary or secondary antibody is chemically coupled to an enzyme, such as horseradish peroxidase or alkaline phosphatase, that converts a colorless substrate into a colored reaction product in situ. The colored product identifying the epigenetic marker may be observed or quantified, such as by spectrometry methods.

In various embodiments, immunoblotting, also called Western blotting, may be used to identify the presence and quantity of an epigenetic marker in a cell lysate. A sample of cells, such as leukocytes, may be solubilized in a detergent to produce free solubilized proteins. The proteins may then be applied to a gel for gel electrophoresis to separate the proteins according to size. The proteins in the gel may be applied to a substrate such as a nitrocellulose membrane. The substrate may be treated with antibodies in which the antibodies bind their specific antigen on the membrane. The epigenetic marker may then be viewed and quantified, such as by using a plate reader.

Similarly to the Western Blot and in accordance with various embodiments, a protein dot blot methodology applies a protein fraction isolated from a cell lysate to a membrane, such as nitrocellulose, in as particular location or “spot.” However, the proteins are not first separated by gel electrophoresis. The protein spot may be treated with a labeled primary or secondary antibody to hybridize the antibody to the antigen, such as an epigenetic marker. Upon development of the fluorescent molecule or colored product identifying, the epigenetic marker, a quantitative measurement can be made of the spots using a spectrometer such as a plate reader.

In addition according to various embodiments, an enzyme-linked immunosorbent assay (ELISA) may be used to detect an antigen, such as an epigenetic marker, using an antibody. To detect the antigen, the sample to be tested, such as a protein fraction from leukocytes, may be coated onto the surface of plastic wells. Labeled antibody, such as a primary or secondary antibody, may be added to the wells under conditions where nonspecific binding is prevented (called “blocking”), such that only binding to the antigen allows the antibody to be retained in the well after washing. The bound antibody may he detected by an enzyme-dependent color change or fluorescent reaction that may be observed and quantified by a spectrometer such as a multiwell plate reader.

Also, in accordance with various embodiments, a high throughput method of quantifying the amount of an epigenetic marker in a biological sample, such as leukocytes isolated from a patient's blood, may comprise flow cytometry, such as fluorescence-activated cell sorting (FACS). Flow cytometry may be used to count the number of immunoreactive cells present in a sample by suspending the cells treated with labeled antibody in a stream of fluid, such as cell culture medium or buffer, and passing the cells by a fluorescence measuring system. The fluorescent properties of each cell may be determined to provide a graph, such as a histogram, indicating the various fluorescence intensities of all the cells in the sample. In one embodiment, threshold values may be applied to determine the presence of a disease state based on the percentage of cells that are immunoreactive in the sample.

Still further and in accordance to various embodiments, an epigenetic marker may be identified in a sample of cells, tissue, or a biological sample by visualization of labeled antibody bound to the epigenetic marker using immunofluorescence microscopy. The sample may be applied to a microscope slide where a primary antibody is applied, such as the antibody diluted in as buffer in which the slide is submerged. Excess primary antibody may be washed away and a labeled secondary antibody may he applied to the slide. The slide may be viewed under a microscope, such as a fluorescence microscope or a confocal fluorescent microscope, configured to emit specific wavelengths of light onto the slide to produce fluorescence. In some embodiments, the intensity of fluorescence may be measured by a detector on the microscope to quantify the intensity of the fluorescence compared to a control sample.

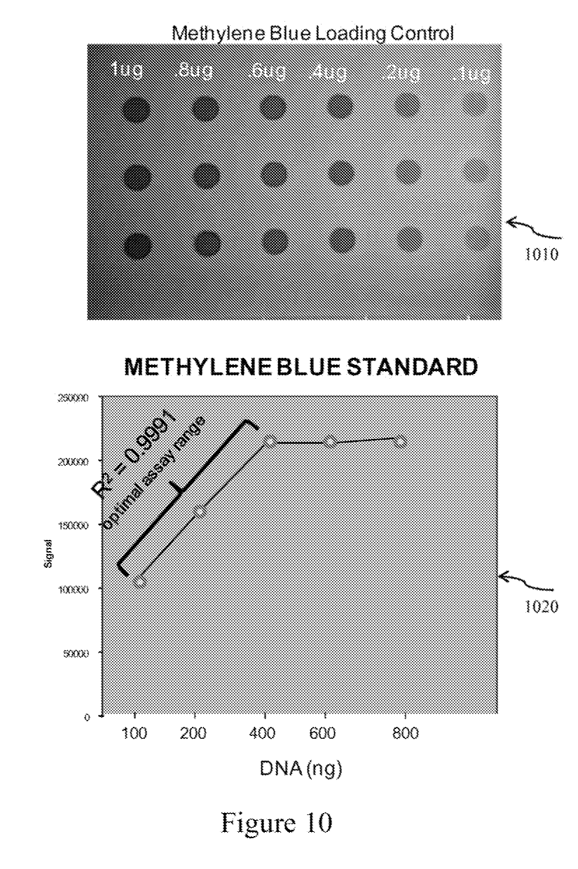

According to various embodiments, methods can include quantifying an amount of an epigenetic marker in a sample. For example, using a quantitative dot blot assay as described herein may be useful for quantitative analysis of an epigenetic marker. With reference to FIG. 10, a diagram illustrating a quantitative dot blot 1010 for methylene blue includes dot blots of 1 μg, 0.8 μg, 0.6 μg 0.4 μg, 0.2 μg, and 0.1 μg. In addition, a calibration curve for methylene blue standard 1020 is also illustrated in FIG. 10. Nitrocellulose membranes of quantitative dot blot 1010 are spotted with various concentrations of DNA (1 μg, 0.8 μg, 0.6 μg, 0.4 μg, 0.2 μg, and 0.1 μg) extracted from blood leukocytes, followed by incubation of the membrane with methylene blue to detect total DNA. Signals are read by standard densitometry. Quantitation of the methylene blue generates the calibration curve for methylene blue 1020.

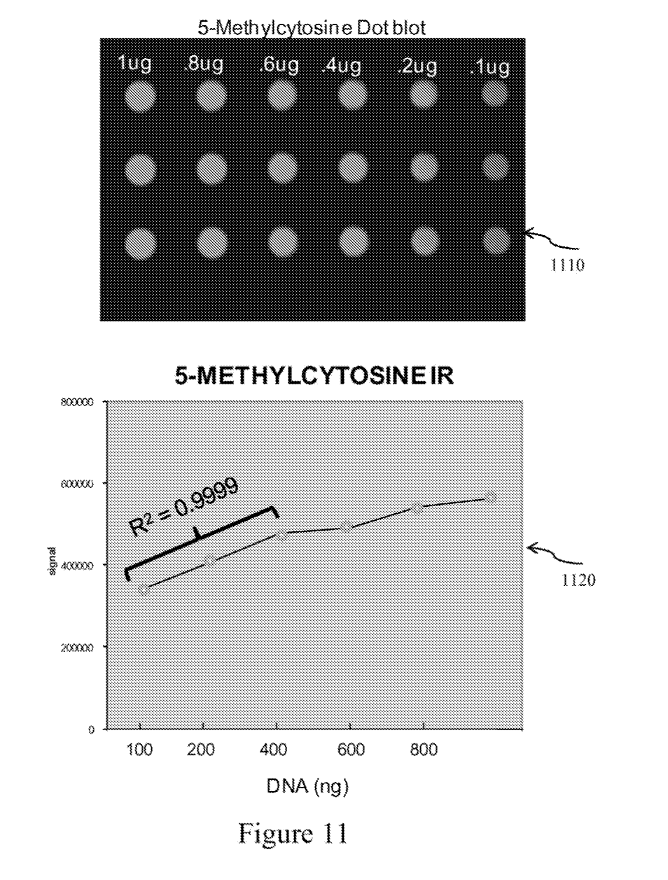

Now with reference to FIG. 11, a diagram illustrating a quantitative dot blot 1030 for 5-methylcytosine includes dot blots of 1 μg, 0.8 μg, 0.6 μg, 0.4 μg, 0.2 μg, and 0.1 μg. In addition, a calibration curve for 5-methylcytosine 1040 is also illustrated in FIG. 11. Nitrocellulose membranes of quantitative dot blot 1010 are spotted with various concentrations of DNA (1 μg, 0.8 μg, 0.6 μg, 0.4 μg, 0.2 μg, and 0.1 μg) extracted from blood leukocytes, followed by incubation of the membrane with 5-methylcytosine to detect total DNA. The 5-methylcytosine signals that are also read by standard densitometry. The quantitation of the methylene blue signal makes it possible to normalize the subsequent 5-methylcytosine reading to the amount of DNA loaded on the blot. Analysis of the signal readings showed that the DNA concentrations from 100 ng to 400 ng gave near linear (R²>99) responses for detecting both DNA and 5-methylcytosine IR content of samples. Identical approaches can be used to develop dot blot assays for other epigenetic markers.

In various embodiments, the present invention provides systems and apparatus that are useful for determining a state of AD in a patient. Accordingly, exemplary systems and/or apparatus can comprise a substrate comprising a top surface and a bottom surface; at least one detail on the top surface of the substrate; at least one antibody operative to bind to at least one epigenetic marker in a sample comprising a leukocyte, the at least one antibody located in the at least one detail; and a reference value, comprising a known amount of the at least one epigenetic marker. These exemplary systems and/or apparatus can further comprise a second detail on the top surface the of the substrate, a second antibody operative to bind a second epigenetic marker in the sample comprising leukocyte, the second antibody can be located in the second detail; and a second reference sample comprising a known amount of the second epigenetic marker. In one embodiment, the at least one detail is a spot and the at least one antibody is bound to the top surface of the substrate. In another embodiment, the at least one detail is a well and the at least one antibody is located in the well. In various embodiments, the sample comprises peripheral blood from a patient. In various exemplary embodiments, the reference value can be located in a reference detail located on the surface of the substrate and proximate to the at least one detail. These exemplary systems and/or apparatus can further comprise a label operable to identify the at least one epigenetic maker. Still further, these exemplary systems and/or apparatus can further comprise a reader operable to measure an amount of the label. The systems and/or apparatus can comprise a cover sealing at least a portion of the top surface of the substrate.

Various embodiments include systems and/or apparatus that comprise a matrix that can detect a plurality of different epigenetic markers from a plurality of sample portions. In an exemplary embodiment, the systems and/or apparatus can further comprise a reference value for each of the plurality of different epigenetic markers. In an aspect of this embodiment, the reference value can be located proximate to the action region of the matrix. In still another embodiment, the systems and/or apparatus can include a calibration curve proximate to each location of the detect plurality of different epigenetic markers. Various embodiments described herein can be adopted for individual home use or in a hospital room or in a doctor's office.

In various embodiments, a kit can comprise an antibody to 5-methylcytosine, peptide involved in DNA methylation, or a peptide involved in histone acetylation, a method to detect binding of the antibody directly (e.g., using primary antibody that is conjugated to a fluorophor, enzyme, or coloring agent) or indirectly (e.g., secondary antibody conjugated to a fluorophor, enzyme, or coloring agent), and at one reference value corresponding to each of thresholds for various diagnoses of AD.

In various embodiments, a kit can comprise a stain or label which can comprise any moiety that can conjugate to an antibody that binds to an epigenetic marker. Still further, in other exemplary embodiments of the kit, the stain or the label can comprise an antibody that hinds to an antibody that binds to an epigenetic marker. Furthermore, a kit can comprise the material to produce a calibration curve for a stain or label, however, the kit may comprise a premade standard calibration which can used as a reference value. The kit can include various buffers and other reagents as described herein. Moreover, a kit can comprise an apparatus or systems described herein. Finally, kits can be designed to be especially useful in one of individual home use or a hospital use or a doctor office, visit

The following are non-limiting examples of various embodiments of the present invention. It should be noted that any combination of materials discussed in these non-limiting examples may be included in a kit, in accordance with various embodiments of the present invention.

### Example 1

### Observation of Global Lyekocyte DNA Methylation Status

7-10 ml of whole blood was collected from 17 living AD and 19 living ND patients in EDTA tubes. White blood cells (WBC) were isolated off of the huffy coat from EDTA whole blood, with RBCs lysed (cold, 1× Lysing solution; NH₄Cl) and 2 subsequent wash cycles with cold. Phosphate Buffered Saline (PBS). The final cell pellet was resuspended in 1 ml cold PBS and 100 μl dropped onto SuperFrost Plus slides and allowed to thoroughly air dry prior to immuncytochemical staining. Some slides were held for as long as one week before staining as this procedure allows for dried, slide storage up to one year.

Slides for 5-methyl cytosine staining were rinsed 3 times, 5 min/each, with cold PBS. The slides are then fixed in 2% paraformaldehyde in PBS) solution for 10 min, followed by rinsing once in PBS and twice in PBS-0.1% TritonX-100 (PBST), each rinse for 10 min/each. The slides were blocked for 30 min in 1% hydrogen peroxide in PBST, followed by 3 PBST rinses as before and then placed into 3% BSA blocking solution (BSA in PBST) for 1 hour at room temperature (RT). This was followed with rinsing one time as before (PBST, 10 min). The slides were then placed in a plastic box and flooded with 5-methyl cytosine antibody at 1:500 in 0.25% BSA-PBST and allowed to stand one hour at RT. The box was then placed into 4° C. overnight, with source of humidity. The following morning the slidebox was removed and allowed to warm to RT. The slides were rinsed 3 times in PBST as before. They were placed into a plastic box and flooded with biotinylated horse, anti-rabbit IgG at 1:1000 in 0.25% BSA-PBST and allowed to stand 2 hours at RT. Slides were rinsed as before with PBST and flood with a prepared Avidin-Biotin solution (in PBST). Following a 45 minute RT incubation, the slides were rinsed once with PBST and then twice with 50 mil Tris (ph 7.6) buffer, 10 min/ea. They were placed into copland jar containing a DAB solution (in 50 mM Tris buffer) for 10 min, RT. Slides were then rinsed twice with iris buffer as before, and then taken through graded alcohols (70%, 90%, 100%, and Neoclear twice) for 5 min/ea, RT. Upon removal from NeoClear and wiping off any excess solution, 2-3 drops of Permount was applied, followed by a coverslip. The slides were allowed to dry at least 2 hours before viewing by bright field microscopy.

Under bright field microscopy, each sample was qualitatively evaluated, as “substantial staining” or “sparse staining”, while blinded to the clinical diagnosis, and evaluated against a qualitative reference of substantial staining in non demented patients. That is samples with spare staining were considered to have Alzheimer's while those with substantial staining were considered to be non-demented. Upon receiving and reviewing the clinical diagnosis for all patients, the evaluation against the reference range agreed with the clinical diagnosis for 16 of the 17 AD cases (94% sensitivity) and 19 of 19 for the non-demented cases (100% specificity)

### Example 2

### Observation of Global Lyekocyte DNA Methylation Mechanisms

7-10 ml of whole blood was collected from 17 living AD and 19 living ND patients in EDTA tubes. White blood cells (WBC) were isolated off of the buffy coat from EDTA whole blood, with RBCs lysed (cold, 1× Lysing solution; NH₄Cl) and 2 subsequent wash cycles with cold Phosphate Buffered Saline (PBS). The final cell pellet was resuspended in 1 ml cold PBS and 100 μl dropped onto SuperFrost Plus slides and allowed to thoroughly air dry prior to immuncytochemical staining. Some slides were held for as long as one week before staining as this procedure allows for dried slide storage up to one year.

Slides for DNMT-1 staining were rinsed 3 times, 5 mm/each, with cold PBS. The slides are then fixed in 2% paraformaldehyde (in PBS) solution for 10 min, followed by rinsing once in PBS and twice in PBS-0.1% TritonX-1.00 (PBST), each rinse for 10 min/each. The slides were blocked for 30 min in 1% hydrogen peroxide in PBST, followed by 3 PBST rinses as before and then placed into 3% BSA blocking solution (BSA in PBST) for 1 hour at room temperature (RT). This was followed with rinsing one time as before (PBST, 10 min). The slides were then placed in a plastic box and flooded with DNMT-1 antibody at 1:500 in 0.25% BSA-PBST and allowed to stand one hour at RT. The box was then placed into 4° C. overnight, with source of humidity. The following morning the slidebox was removed and allowed to warm to RT. The slides were rinsed 3 times in PBST as before. They were placed into a plastic box and flooded, with biotinylated horse, anti-rabbit IgG at 1:1000 in 0.25% BSA-PBST and allowed, to stand 2 hours at RT. Slides were rinsed as before with PBST and flood with a prepared Avidin-Biotin solution (in PBST). Following a 45 minute RT incubation, the slides were rinsed once with PBST and then twice with 50 mM Tris (ph 7.6) butler, 10 min/ea. They were placed into copland jar containing a DAB solution (in 50 mM Tris buffer) for 10 min, RT. Slides were then rinsed twice with Tris buffer as before, and then taken through graded alcohols (70%, 90%, 100%, and NeoClear twice) for 5 min/ea, RT. Upon removal from NeoClear and wiping off any excess solution, 2-3 drops of Permount was applied, followed by a coverslip. The slides were allowed to dry at least 2 hours before viewing by bright field microscopy.

Under bright field microscopy, each sample was qualitatively evaluated as “substantial staining” or “sparse staining”, while blinded to the clinical diagnosis, and evaluated against a qualitative reference of substantial staining in non demented patients. That is samples with sparse staining were considered to have Alzheimer's while those with substantial staining were considered to be non-demented. Upon receiving and reviewing the clinical diagnosis for all patients, the evaluation against the reference range agreed with the clinical diagnosis for 16 of the 1.7 AD cases (94% sensitivity) and 19 of 19 for the non-demented cases (100% specificity).

### Example 3

In accordance with an exemplary embodiment of the present invention, a dot blot method for overall DNA methylation is described. Hybond-ECL nitrocellulose membrane is pre-wet with 6×SSC buffer, and DNA samples are denatured by adding 0.4 M NaOH followed by heating to 100° C. for 10 min. Neutralization of the DNA solution is by addition of 2 M ammonium acetate, pH 7.0. After rehydration of the membrane with 500 μl dH₂O and placement in the dot blot manifold (Gibco), 200-500 μl of the denatured DNA sample is added and pulled through the membrane by gentle vacuum or gravity filtration. To each well, 500 μl of 2×SSC buffer is then added, and vacuum is applied. When the sample wells are empty, the membrane is removed, air-dried for 1 hour at RT, placed between 2 sheets of filter paper, and baked under vacuum at 80° C. for 2 hours. To probe for CpG methylation, the membrane is blocked lot 2 hours in 5% milk in dot blot buffer (20 mM Tris, 0.05% Tween-20), then washed 1× in dot blot buffer. Incubation with 5-methylcytosine arouse monoclonal primary antibody (Aviva Systems Biology), diluted 1:1000, is for 2 hours at RT in dot blot buffer and 5% milk. After washing 5× (5 min each), the membrane is incubated with 1:5000 HRP-conjugated mouse monoclonal secondary antibody (Jacksons immuno) for 1 hour at RT in dot blot buffer and 5% milk, followed by 5N washing (5 min each). The membrane is then incubated for 1 min at RT with Super Signal West Pico Chemiluminescence Substrate (Thermo Scientific), imaged on an Alpha Innotech AlphaEase instrument, and analyzed using Alpha Innotech FC software. All readings are normalized to methylene blue standards, prepared by washing the membrane (after the 5-methylcytosine data are collected) with dH₂O to remove Chemiluminescence substrate, incubation with 0.025% methylene blue for 1 min, washing 1× with dH₂O, and quantitation by AlphaEase using FC software. In addition to this novel method of measuring overall DNA methylation using a DNA substrate, standard dot blots for measurement of peptides can be used to quantify other epigenetic markers such as HDAC1 and DNMT1. For the latter, similar methods are employed, except that protein extracts are loaded instead of DNA, and antibodies appropriate to the peptides are utilized for detection

In the foregoing specification, the method and system to detect, diagnose, and monitor the progression of AD has been described with reference to specific embodiments. Various modifications and changes may be made, however, without departing from the scope of the method and system to detect, diagnose, and monitor the progression of AD as may be set forth in the claims. The specification and figures are illustrative, rather than restrictive, and modifications are intended to be included within the scope of the method and system to detect, diagnose, and monitor the progression of AD. Accordingly, the scope of the method and system to detect, diagnose, and monitor the progression of AD should be determined by the claims and their legal equivalents rather than by merely the examples described.

Benefits, other advantages and solutions to problems have been described with regard to particular embodiments; however, any benefit, advantage, solution to problem or any element that may cause any particular benefit, advantage or solution to occur or to become more pronounced are not to be construed as critical, required or essential features or components of any or all the claims in any issuing patent.

The terms “comprise”, “comprises”, “comprising”, “having”, “includes” and the like refer to a non-exclusive inclusion, such that a process, method, system, article, composition or apparatus that comprises a list of elements does not include only those elements recited, but may also include other elements not expressly listed or inherent to such process, method, system, article, composition or apparatus. Other combinations and/or modifications of the structures, arrangements, applications, proportions, elements, materials or components used in the practice of the method and system to detect, diagnose, and monitor the progression of AD, in addition to those not specifically recited, may be varied or otherwise particularly adapted to specific environments, manufacturing specifications, design parameters or other operating requirements without departing from the general principles of the same.

All literature and similar materials cited in this application, including but not limited to, patents, patent applications, articles, books, treatises, and internet web pages, regardless of the format of such literature and similar materials, are expressly incorporated by reference in their entirety for any purpose. In the event, that one or more of the incorporated literature and similar materials differs from or contradicts this application, including but not limited to defined terms, term usage, described techniques, or the like, this application controls.

1. A method for diagnosing Alzheimer's disease in a patient, the method comprising:
placing a sample comprising at least one blood component onto a substrate, wherein the at least one blood component comprises at least one of a white blood cell or a protein-containing extract of the white blood cell;
labeling the sample with a label to identify at least one of a DNA methylation marker and a histone modification marker that change as a characteristic of Alzheimer's disease;
determining an amount of a signal from the label, wherein the amount of the signal from the label correlates to the amount of the at least one DNA methylation marker and histone modification marker;
comparing the amount of the sign al from the label to a control; and
determining the diagnosis of Alzheimer's disease based on the comparison of the amount of the signal from the label to the control, wherein a decrease in the amount of the signal from the label compared to the control indicates a diagnosis of Alzheimer's disease.

2. The method according to claim 1, wherein the at least one blood component is purified from whole blood.

3. The method according to claim 1, wherein the determining the amount of the signal from the label comprises measuring an intensity of the signal from the label.

4. The method according to claim 1, further comprising binding an antibody to the at least one DNA methylation marker and histone modification marker.

5. The method according to claim 1, further comprising preparing a treatment plan for a patient supplying the sample.

6. The method according to claim 5, further comprising treating the patient with a therapeutic substance.

7. The method according to claim 6, further comprising:
placing a second sample comprising the at least one blood component onto the substrate;
labeling the second sample to identify the at least one epigenetic marker;
determining a second amount of the at least one epigenetic marker;
comparing the second amount to the reference value; and
determining a dosage of the therapeutic substance.

8. The method according to claim 6, further comprising evaluating the efficacy of the therapeutic substance.

9. The method according to claim 8, wherein the evaluating, the efficacy of the therapeutic substance further comprises comparing a plurality of epigenetic markers over time.

10. The method according to claim 1, further comprising estimating a likelihood that the patient will develop clinical signs of Alzheimer's disease based on the comparison of the amount of the signal from the label to a second control, wherein the second control comprises an amount of a second control signal from the label identifying an amount of the at least one DNA methylation marker and histone modification marker from a second sample comprising at least one blood component from the same patient collected, at an earlier point in time than when the sample was collected from the patient, wherein a decrease of the signal as compared to the second control signal indicates an increase in the likelihood that the patient will develop clinical signs of Alzheimer's disease.

11. The method according to claim 10, wherein the estimating the likelihood further comprises comparing a plurality of it patient's at least one DNA methylation marker and histone modification marker amounts over time.

12. The method according to claim 1, further comprising:
labeling the sample with a second label to identify a second at least one DNA methylation marker and historic modification marker;
determining an amount of a second signal from the second label, wherein the amount of the second signal from the second label correlates to an amount of the second at least one DNA methylation marker and historic modification marker;
comparing the amount of the second signal from the second label to a second control; and
further diagnosing Alzheimer's disease based on the comparison of the amount of the second signal from the second label to the second control.

13. A method for diagnosing Alzheimer's disease in a patient, the method comprising:
receiving, a blood sample from the patient;
separating leukocytes from the blood sample;
binding an antibody to at least one DNA methylation marker and historic modification marker that change as a characteristic of Alzheimer's disease in the leukocytes;
attaching a label to the antibody;
quantifying an amount of a signal from the label attached to the antibody, wherein the amount of the signal from the label attached to the antibody correlates to the amount of the at least one DNA methylation marker and historic modification marker;
comparing the amount of the signal from the label to a control; and
diagnosing Alzheimer's disease in the patient based on the comparison of the amount of the signal from the label to the control, wherein the diagnosis of Alzheimer's disease is determined when there is a decrease of immunoreactivity of the at least one DNA methylation marker and histone modification marker to the antibody in the blood sample relative to the immunoreactivity of the at least one DNA methylation marker and historic modification marker to the antibody in the control.

14. The method according to claim 13, further comprising:
binding a second antibody to a second at least one DNA methylation marker and histone modification marker in the leukocytes;
attaching a second label to the second antibody;
quantifying an amount of a signal from the second label, wherein the amount of the signal from the second label correlates to the amount of the second at least one DNA methylation marker and historic modification marker; and
further diagnosing Alzheimer's disease in the patient based on comparing the amount of the signal from the label and the amount of the signal from the second label.

15. The method according to claim 13, wherein the signal from the label attached to the antibody is quantified by plotting the signal's intensity on a calibration curve comprising signal intensity of known amounts of unbound label.

16. The method according to claim 13, wherein the attaching a label to the antibody comprises conjugating an antibody comprising the label to the antibody.

17. A method for diagnosing Alzheimer's disease in a patient, the method comprising:
placing a sample comprising at least one blood component onto a substrate, wherein the at least one blood component comprises at least one of a white blood cell or a protein-containing extract of the white blood cell;
labeling the sample with at least a first label to identify at least one DNA methylation marker, and labeling the sample with at least a second label to identify at least one histone modification marker, wherein the at least one DNA methylation marker and the at least one histone modification marker change as a characteristic of Alzheimer's disease;
determining an amount of a first signal from the first label, wherein the amount of the first signal from the first label correlates to the amount of the at least one DNA methylation marker, determining, an amount of a second signal from the second label, wherein the amount of the second signal from the second label correlates to the amount of the at least one histone modification marker;
comparing the amount of the first signal from the first label to a first control, and comparing the amount of the second signal from the second label to a second control; and
determining the diagnosis of Alzheimer's disease based on the comparison of the amount of the first signal from the first label to the first control, and comparing the amount of the second signal from the second label to a second control, wherein the diagnosis-of Alzheimer's disease is determined when the amount of the first signal is decreased as compared to the first control and the amount of the second signal is decreased as compared to the second control.

18. The method of claim 17, wherein the patient is alive.

19. The method according to claim 17, wherein the determining the amount of the first signal from the first label and the amount of the second signal from the second label comprises measuring an intensity of the first label and the second label.
